(19) United States
(12) Patent Application Publication
(10) Pub. No.: US 2023/0412593 A1
(43) Pub. Date: Dec. 21, 2023

(54) Device Component of Digital Healthcare Platform

(71) Applicant: Health2047, Inc., Menlo Park, CA (US)

(72) Inventors: Charles Edward George Keith Aunger, Hayward, CA (US); Judy L. Barkal, Menlo Park, CA (US); Gloria N. Cao, San Jose, CA (US); Edison Ting, Celina, TX (US)

(21) Appl. No.: 18/198,665

(22) Filed: May 17, 2023

Related U.S. Application Data
(60) Provisional application No. 63/343,069, filed on May 17, 2022.
(60) Provisional application No. 63/343,071, filed on May 17, 2022.
(60) Provisional application No. 63/343,072, filed on May 17, 2022.
(60) Provisional application No. 63/343,073, filed on May 17, 2022.

(51) Int. Cl. H04L 9/40; H04L 9/30

(52) U.S. Cl. CPC H04L 63/0861; H04L 2209/88; H04L 9/3066; H04L 63/0876

(57) ABSTRACT

A novel device component in a digital healthcare framework adds context to physiological sensor data, where such context data includes a first unique identifier associated with the device and a second unique identifier associated with the user who the physiological data belongs to. The physiological sensor data along with the context data is then encrypted and transmitted via an encrypted channel to a coordination gateway.

### CROSS-REFERENCE TO RELATED APPLICATIONS

The present application claims the benefit of U.S. Provisional Application No. 63/343,069 filed on May 17, 2022; the entire disclosure contents are fully incorporated by reference into the present application. The present application claims the benefit of U.S. Provisional Application No. 63/343,071 filed on May 17, 2022; the entire disclosure contents are fully incorporated by reference into the present application. The present application claims the benefit of U.S. Provisional Application No. 63/343,072 filed on May 17, 2022; the entire disclosure contents are fully incorporated by reference into the present application. The present application claims the benefit of U.S. Provisional Application No. 63/343,073 filed on May 17, 2022; the entire disclosure contents are fully incorporated by reference into the present application.

Applicant of the present application owns U.S. application Ser. No. 17/446,210 filed on Aug. 27, 2021, entitled “Enhanced Authentication Techniques Using Virtual Persona,” which is herein fully incorporated by reference in its entirety.

### BACKGROUND OF THE INVENTION

### Field of Invention

The present invention generally relates to healthcare platforms. More specifically, the present invention is related to a novel device component of a digital healthcare platform.

### Discussion of Related Art

FIG. 1 depicts the current state-of-the-art in remote patient care. Element 102 represents a medical device (e.g., a blood pressure monitor, a glucometer, or other medical devices) that is configured to collect physiological data associated with a patient. In current state-of-the-art systems, physiological data is measured (via one or more sensors on the medical device) at the client-side (i.e., the patient side) and is transmitted, typically over the Internet, via an encrypted channel 104 to a data platform at a remote location. The data platform logs the received data in a logs database 106.

In current state-of-the-art systems, data that gets sent from the client-side typically comprises a unique device identifier (ID) in addition to the physiological data. For example, in the case of a blood pressure monitor, the client-side device transmits systolic/diastolic pressures, along with additional information such as pulse rates, etc. When such information is received by the data platform, it logs this information into the logs database 106. In this setup, an additional correlation or mapping is needed to associate the data received and stored with the device ID identifying the specific device that transmitted the data and a user identifier (ID) identifying which user is associated with the transmitted data. Data identifying which device transmitted the data is stored in a Device IDs database 108 and data identifying which user is associated with the transmitted data is stored in a User IDs database 110.

As one example, a first table may be maintained that maps a second table of device IDs and a third table of user IDs, where the data platform performs a look-up of a particular user ID to determine a corresponding device ID (device IDs if there are a plurality of devices associated with a particular user) or may perform a look-up of the device ID to determine the associated user ID. Accordingly, when the clinician accesses the data platform (via, for example, an application such as a patient dashboard) to review physiological data associated with a given patient, the data platform (at the back-end) correlates the user ID (obtained from the User IDs database 110) of the given patient with the device ID of the given patient (obtained from the User IDs database 108), retrieves data associated with the given patient based on such correlation, and renders such retrieved data at the clinician's end. In such state-of-the-art systems, a lot of time/resources are spent in resolving such correlations so that the physician views data associated with the appropriate patient that he/she is requesting data for.

Also, in such state-of-the-art systems, much of the correlation/mapping (or steps leading up to the correlation/mapping) is performed after all of the device data is sent over to the data platform. As a result of this, some of the client-side information gets lost. For example, by the time the device data is sent to the data platform, the specific person taking the measurement is lost as that data is not sent to the data platform along with the physiological data collected by the device. This leads such state-of-the-art systems to maintain mapping/correlation data after the physiological data is collected, which may not be desirable.

Another problem with such state-of-the-art systems is that they are unable to distinguish who specifically in a household used the device. Clinicians experience problems when they do not know who was using the device at the time physiological data was collected, as there may have been additional members of the household that may have had access to the same device. A workaround that may be used is to check if measurements deviate from historical data, but such workarounds are inherently not accurate. Likewise, having clinicians look for such deviations also disrupts their actual workflow as they are no longer focused on analyzing patient data, but are rather focused on whether or not the patient data corresponds to the right person in the household (especially in instances where collected physiological data shows a large deviation).

Further, in the state-of-the-art set up, the services that the clinician provides are billable. Accordingly, the time the clinician spends reviewing the data and/or talking with the patient is billable through a central location such as the Centers for Medicare and Medicaid Services (CMS).

There is, thus, a billing component where the data (that is collected in the data platform) and transactions associated with a patient (in terms of the time the physician is spending with the patient and the time the physician is spending reviewing the data from the devices associated with that patient) are used in processing claims, usually by a claims department.

Such claims departments use codes called Current Procedural Terminology (CPT®) codes, where each CPT® code is a five-digit numeric code assigned to each task and service a healthcare provider offers (e.g., medical, surgical, and diagnostic services). Insurers use the numbers to determine how much money to pay a physician (e.g., certain CPT® codes allow a physician to bill 20 or 40 minutes of his/her time). Such CPT® codes may be stored in a CPT® codes database 112. During claims processing, a determination needs to be made regarding what data may be mapped to which CPT® code, and from that determine what the billable details are.

Another important code is the International Classification of Diseases (ICD©) code. The World Health Organization (WHO) defines ICD-11© as a classification and terminology system that “allows the systematic recording, analysis, interpretation and comparison of mortality and morbidity data collected in different countries or regions and at different times” and “ensures semantic interoperability and reusability of recorded data for the different use cases beyond mere health statistics, including decision support, resource allocation, reimbursement, guidelines and more.” Broadly, ICD-11© codes refer to the condition being treated (i.e., high blood pressure, diabetes, etc.). ICD-11© codes ensure that a patient gets proper treatment and is charged correctly for any medical services they receive. ICD-11© codes are used in billing (where billing is based on rules stored in a billing rules databases 116), treatments, and statistics collection. Having the right code is important to ensure that standardized treatment for a medical issue is delivered and that medical expenses are reimbursed. Such ICD-11© codes may be stored in an ICD-11© codes database 114. When a healthcare provider submits a bill to an insurance company for reimbursement, each service is described by the CPT® code, and this CPT® code is matched to an ICD-11© code. If the two codes don't align correctly with each other, the insurance company may deny payment.

One problem with CPT® codes is that a plurality of CPT® codes may apply to the

same data set. As an example, a first patient may send data in digital form, while the same patient may send another set of data by manually entering it. It happens that those two mediums (i.e., digital vs manual) apply to two different CPT® codes, and they have different billing requirements. While the clinician just wants to see the data and not necessarily the billing codes and the manner in which the data was sent, often times the claims department has to consult back with the clinician to see if this is digitally or manually collected. In many instances, the software development has to go in and determine how it was collected.

Another problem with standardized codes, such as CPT® codes, (while not knowing the codes beforehand at the edge or client-facing application and having to find the mapping later at the backend/data platform side) is that rules that apply for CPT® codes tend to change from time to time. For example, let's take code 99454 (a billing code for supplying and monitoring patients with remote patient monitoring devices) as an example. A couple of years ago, the rule was that each patient should at least submit two data points in a month at a minimum (as long as someone reviewed the data), which allows the physician providing the service to that patient to bill for that CPT® code. However, it was recently revised to state that the same CPT® code now requires the patient to submit 16 data points in a month. State-of-the-art systems lack sophistication in determining what rules apply to data collected prior to a rule change and what rules apply for data collected since the rule has gone into effect.

Such state-of-the-art healthcare frameworks also do not provide an easy manner in which to incorporate new data sources (i.e., physiological data from non-clinically validated devices) without adding more complexity. Yet data sources from these devices could provide supporting evidence that may be useful for the health care team.

Such problems with state-of-the-art systems cause a lot of inefficiencies and problems in terms of the way information is processed, and such inefficiencies and problems lead to various errors.

Embodiments of the present invention are an improvement over prior art systems and methods.

### SUMMARY OF THE INVENTION

In one embodiment, the present invention provides a medical device comprising: (a) a sensor configured to collect physiological data; (b) an authentication engine, the authentication engine configured to: (1) receive a unique device identifier (UDI), the UDI uniquely identifying the medical device; (2) receive a unique user identifier (UUI), the UUI uniquely identifying a user of the medical device; (3) authenticate the user as an authorized user of the medical device based on the UDI and the UUI; (4) upon successful authentication, formulate a data frame with: (i) collected physiological data, (ii) UUI, and (iii) UDI; (c) an encryption engine encrypting the data frame via an encryption algorithm; and (d) a transmission component transmitting the encrypted data frame.

In another embodiment, the present invention provides a method as implemented in a medical device, the method comprising: (a) collecting physiological data; (b) receiving a unique device identifier (UDI), the UDI uniquely identifying the medical device; (c) receiving a unique user identifier (UUI), the UUI uniquely identifying a user of the medical device; (d) authenticating the user as an authorized user of the medical device based on the UDI and the UUI; (e) upon successful authentication, formulating a data frame with: (1) collected physiological data, (2) UUI, and (3) UDI; (f) encrypting the data frame via an encryption algorithm; and (g) transmitting the encrypted data frame.

In yet another embodiment, the present invention provides an article of manufacture comprising non-transitory computer storage medium storing computer readable program code which, when executed by a computer, implements a method as implemented in a medical device, the medium comprising: (a) computer readable program code collecting physiological data; (b) computer readable program code receiving a unique device identifier (UDI), the UDI uniquely identifying the medical device; (c) computer readable program code receiving a unique user identifier (UUI), the UUI uniquely identifying a user of the medical device; (d) computer readable program code authenticating the user as an authorized user of the medical device based on the UDI and the UUI; (e) computer readable program code, upon successful authentication, formulating a data frame with: (1) collected physiological data, (2) UUI, and (3) UDI; (f) computer readable program code encrypting the data frame via an encryption algorithm; and (g) transmitting the encrypted data frame.

### BRIEF DESCRIPTION OF THE DRAWINGS

The present disclosure, in accordance with one or more various examples, is described in detail with reference to the following figures. The drawings are provided for purposes of illustration only and merely depict examples of the disclosure. These drawings are provided to facilitate the reader's understanding of the disclosure and should not be considered limiting of the breadth, scope, or applicability of the disclosure. It should be noted that for clarity and ease of illustration these drawings are not necessarily made to scale.

FIG. 1 depicts the current state-of-the-art in remote patient care.

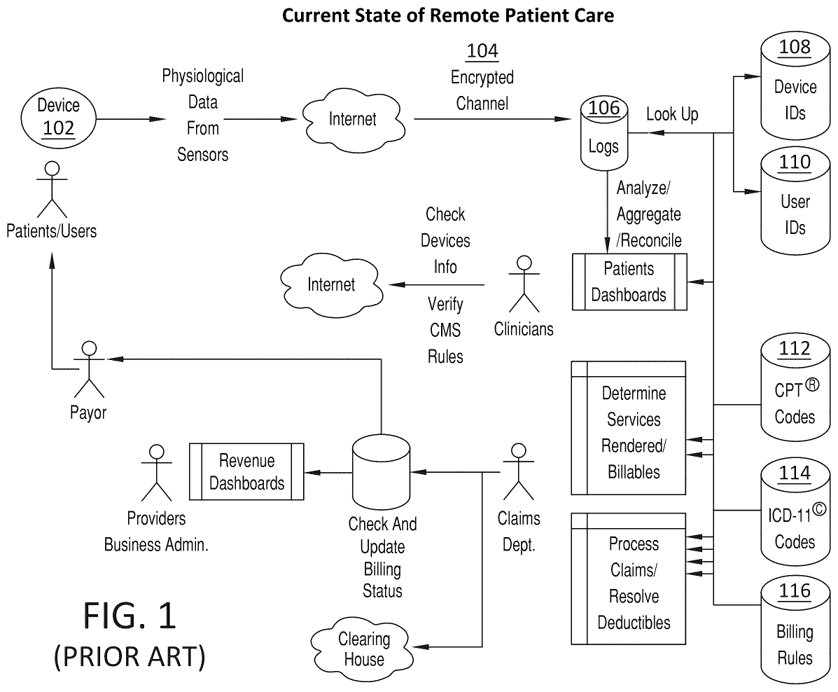

FIG. 2 depicts an overview of the present invention's digital healthcare framework.

FIG. 3 depicts a method as implemented in the device component of the digital healthcare framework.

### DESCRIPTION OF THE PREFERRED EMBODIMENTS

While this invention is illustrated and described in a preferred embodiment, the invention may be produced in many different configurations. There is depicted in the drawings, and will herein be described in detail, a preferred embodiment of the invention, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the present invention.

Note that in this description, references to “one embodiment” or “an embodiment” mean that the feature being referred to is included in at least one embodiment of the invention. Further, separate references to “one embodiment” in this description do not necessarily refer to the same embodiment; however, neither are such embodiments mutually exclusive, unless so stated and except as will be readily apparent to those of ordinary skill in the art. Thus, the present invention can include any variety of combinations and/or integrations of the embodiments described herein.

The present invention provides a digital healthcare framework that ensures trust and integrity of the data, and enables true ownership of data, defining which patient the data corresponds to and what are the permitted uses for that data. In the present invention's digital healthcare framework, substantial work is performed at the edge (i.e., the client or patient side) to determine: (1) what data is collected by the sensors and what is the context of the data collected, (2) who was using the device, and (3) what rules apply to the collected data. The present invention's digital healthcare framework, at the highest level, provides for streaming data collected at the edge (i.e., the client or patient side) in a more intelligent way by attaching a context to that data, as will be explained below.

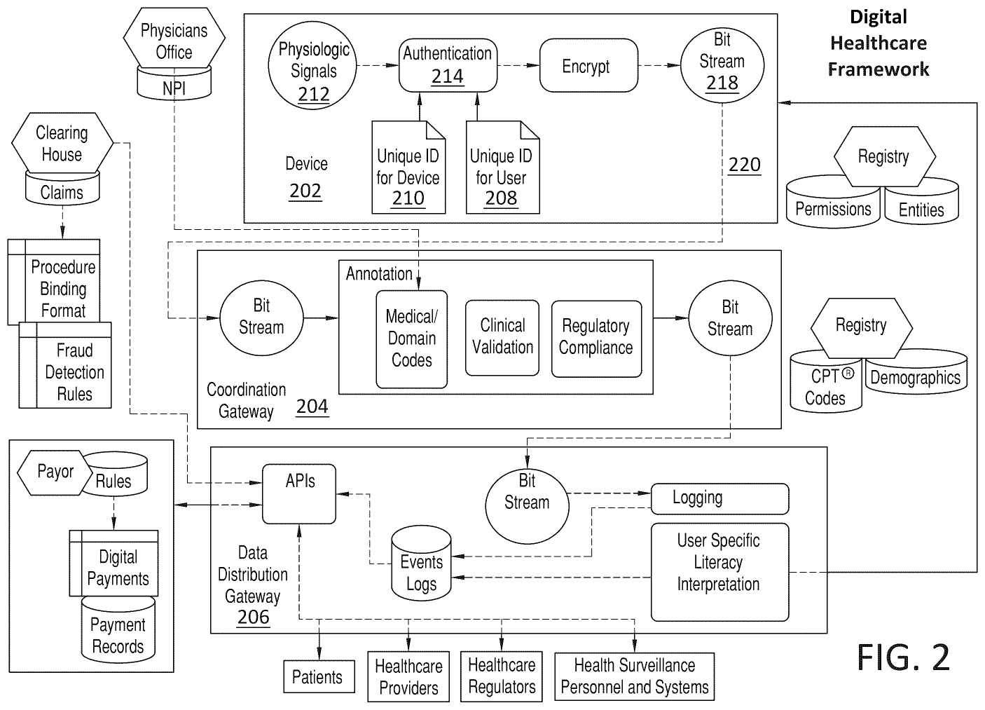

FIG. 2 depicts an overview of the present invention's digital healthcare framework 200 comprising the following components: (1) device component 202; (2) coordination gateway component 204, and (3) data distribution gateway component 206. The present patent application focuses on the novelty aspects of the device component 202. Additional information on the functionality associated with coordinating gateway 204 may be found in the concurrently filed non-provisional patent application titled “Coordinating Gateway Component of Digital Healthcare Platform”, with U.S. Serial No. XX/XXX,XXX, which is fully incorporated by reference in its entirety. Additional information on the functionality associated with data distribution gateway 206 may be found in the concurrently filed non-provisional patent application titled “Data Distribution Component of Digital Healthcare Platform”, with U.S. Serial No. XX/XXX,XXX, which is fully incorporated by reference in its entirety.

Device 202 represents a medical device (e.g., a blood pressure monitor, a glucometer, oximeter, spirometer, oxygen nebulizer, stethoscope, electrocardiogram (ECG) device, thermometer, activity tracker, weighing scale, or other medical devices) that is configured to collect physiological data associated with a patient. It should be noted that while specific examples of devices such as blood pressure monitors, glucometers, etc., are used throughout the specification, these are non-limiting examples of such devices. Any device that is capable of collecting and transmitting (to either a remote location by itself or via a proxy device) one or more physiological data points is envisioned for use with the present invention.

Unlike the state-of-the-art systems, in the digital healthcare framework of FIG. 2, a unique user identifier (ID) 208 based on the identity of the user of the device 202 and a unique device identifier (ID) 210 that uniquely identifies the device are assigned as context to the physiological data collected at the device 202. In one example, the device itself could provide a unique identifier that may be “hardcoded” or encoded through SIM or eSIM type programmable technology. In another example, the identifier may be associated with the hardware within the device (e.g., model number, serial number, etc.), or may be associated with software configurations of the device, or both. The device identifier may include a Host ID assigned to it by a registry function of the digital healthcare platform that permits it to communicate on the same platform. Other non-limiting examples of context data associated with the device include: date, time, and other location and environmental data (e.g., temperature, humidity, air, etc.).

According to the present invention, physiological sensor data 212 is collected by device 202, but before the device sends the collected physiological sensor data 212, the device attaches to a data frame (having data associated with the collected physiological sensor data 212), information about “who” (i.e., which user was using device 200 when the collected physiological sensor data 212 was collected) and “what” (i.e., what device is collecting the physiological sensor data 212). The data frame noted above is a message stack that is transmitted as an encrypted stream.

The authentication engine 214 addresses the “who” aspect above by authenticating the user prior to data collection/data transmission, where such features are implemented and performed at the device level. Once the user is identified by the authentication engine 214, a unique user ID 208 is identified for attaching to a data frame. The authentication data is part of the transmitted data and could be temporarily stored on the device if needed. The device performs a service request to the authentication component to get/update the authentication information.

In one example, a user may be a user of the medical device and may be required to provide authentication information to the medical device. Example authentication information may include a username and password, biometric authentication information, a keycard, a badge, etc. Thus, the user may be required to input authentication information prior to accessing the medical device. The above-described authentication information may be used to confirm that the user is who they say they are. That is, the authentication information may be indicative of the identity of the user who is attempting to use the medical device.

However, a password may be improperly obtained by a malicious actor. Thus, it may be advantageous to add an extra layer of security onto solely requiring a password. Different techniques may be employed for this extra layer. As an example, when the user provides his/her password to the medical device, the user's mobile device may receive information identifying a unique number. For example, the unique number may be provided as a text message to the mobile device or may be presented via an application executing on the user's device. This added technique, known as two-factor authentication, may allow for an increase in confidence in the identity of the user. For example, it is more likely that the user is a specific person if: (1) the user knows the person's password and (2) has access to the unique number.

Additionally, or alternatively to the above, biometric authentication may be used to increase confidence in the identity of the user. For example, a camera may obtain an image of the user's face. The resulting image may be analyzed to determine whether it reflects a specific person. As an example, a neural network may be used to generate an encoding of the face into a learned vector space. A distance metric may be computed between the encoding and encodings known to be associated with specific persons. In this way, a person who corresponds to the face in the photo may be identified. Additional biometric authentication may include a thumbprint, fingerprint, voice signature, and so on.

In addition to the above-described authentication techniques, a virtual persona may be maintained for a user. The virtual persona may be associated with information usable to uniquely confirm the identity of the user. For example, and as may be appreciated, a person may typically have certain states in his/her daily life. Example states may include being at home, being at work, being in transit (e.g., commuting), engaging in weekend activities, engaging in vacations, and so on. For each state, there may be a combination of features or information which can be used to determine an identity of a corresponding person.

As an example, with respect to being at home, a person may typically use a set of devices while at home. For example, the person may typically access a specific mobile device to perform certain actions (e.g., read the news, message friends, and so on). As another example, the person may typically activate a smart television (TV) device to watch streaming media, television, etc. As another example, the person may typically wear a specific wearable device (e.g., smartwatch) while in his/her home. As another example, the person's mobile device may communicate with certain smart speakers, an intelligent personal assistant executing on a smart speaker, and so on.

In the above-described example, the collection of devices may be used to identify the person uniquely. For example, it may be unlikely that a different person may have access to the same mobile device. In this example, the mobile device may be identified based on unique identifying information such as a media access control (MAC) address, an international mobile equipment identity (IMEI) code, and so on. For example, a hash of the identifying information may be stored. Additionally, it may be even less likely that a different person may have access to the same mobile device and also access to the same smart TV device, specific wearable device, and so on. Thus, if all of these devices are determined to be proximate to a person, then a reasonable determination that the person corresponds to a specific identity may be effectuated.

In addition to devices, further information may be used to increase confidence in the above-described person's identity. For example, the behavior of the person may be monitored over time. In this example, the person may typically follow a certain schedule. An example schedule may include the person being at home from certain hours of the day (e.g., 6 pm to 8 am). While the person is at home, the person may use the devices described above. The example schedule may then indicate that the person drives to work, takes the subway to work, and so on. During this commute, the person may be proximate to devices that are substantially similar across different days. For example, the person may be proximate to a car stereo that is uniquely identifiable. As another example, the person may be proximate to a Wi-Fi access point which is uniquely identifiable via an access point name (APN). As another example, during the commute, the person's location may change. For example, the person's mobile device may record changing locations. As another example, the person's mobile device may connect to different cell towers as the location changes. In the above example, such behavior may be determined to correspond with a specific identity very accurately. Indeed, and as may be appreciated, as the features of a person are more closely monitored, confidence in the person's identity may be increased.

The above-described information may thus form a virtual persona for the person and be monitored by one or more controllers associated with the person. For example, and as described above, a controller may represent a mobile device used by the person. The controller may execute an application that enables the gathering and monitoring of at least the above-identified information. Thus, when the person attempts to access a system or software, the controller may automatically provide authentication information to the system or software. With respect to the medical device, such a controller may provide authentication via a wireless or wired connection. Since the controller monitors the virtual persona, the controller may determine to a substantially high confidence metric that the person is authorized to use the medical device. As described above, each device may represent a feature. An aggregation of these features, such as a threshold amount, may be used to identify the identity of the user (referred to as a ‘master mesh’). As described herein, each mesh may be associated with example information associated with the example user. The example information may be used to confirm an identity associated with the example user. For example, when the example user attempts to access an application, software, system, and so on, the virtual persona may be used to confirm the identity of the user. Also, as an example, the aggregated amount may represent an amount that is presently proximate to the smartphone, or which was proximate within a threshold amount of time, or at respective times known to be commonly proximate to the smartphone.

Additional specific examples of virtual personas, meshes, and authentication techniques may be found in Applicant-owned U.S. application Ser. No. 17/446,210, which is fully incorporated by reference in its entirety.

Physiological data is measured (via one or more sensors on device 202) at the client-side (i.e., the patient side), and context information such as unique device ID 210 and unique user ID 208 are added to such physiological data, where collected data is enhanced with context information. Such enhanced data is then encrypted, via encryption engine 216, to form bitstream 218 which is typically transmitted over the Internet, via an encrypted channel 220 to a coordination gateway 204 at a remote location. In one non-limiting example, it is envisioned that the host identity protocol (HIP) could be the identification technology that is used in the digital health platform.

In such an example, the paper to Moskowitz et al. titled, “New Cryptographic Algorithms for HIP” (Internet-Draft: draft-moskowitz-hip-new-crypto-06), which is fully incorporated by reference, outlines examples of cryptographic algorithms that may be used with HIP. Non-limiting examples of cryptographic algorithms for HIP include: new elliptic curves for Elliptic-curve Diffie-Hellman (ECDH), the Edwards Elliptic Curve Digital Signature Algorithm (EdDSA) used in Host Identities (HI) and for Base Exchange (BEX) signatures, hashes used in Host Identity Tag (HIT) generation, and wherever else hashes are needed, keyed hashes used for KEYMAT (Keying Material) generation and packet MACing (Message Authentication Code) operations, and Authenticated Encryption with Associated Data (AEAD) and stream ciphers to use in Host Identify Protocol (HIP) and HIP enabled secure communication protocols.

The registry depicted in FIG. 2 allows users, devices, organizations/entities, authorized people, etc. to register so they can use the digital health framework. Each user type has an encrypted host identifier and is assigned permissions for what they are allowed to do on the network (similar to role-based permissions). Some permissions are government regulated (e.g., HIPAA), and others are based on licenses (e.g., Current Procedural Terminology or CPT®).

Another key component of device component 202 is the ability to provide feedback to device component 202 such that it is informed of various pieces of information. The distribution gateway component 206 includes a user-specific interpretation component that provides such feedback to device component 202 where such feedback may be rendered to the user via a variety of mechanisms. For example, device component 202 may include a display (not shown) that displays such feedback information, or device component 202 may include a speaker (not shown) to announce such feedback information. Alternatively, device component 202 may provide such information to another external device, such as but not limited to, a smartphone device or a tablet device, to render such information (e.g., display such information or announce such information) on such an external device. Additionally, it is also envisioned that such device components also are able to render such feedback information in a multilingual format or render such feedback in a modified manner (e.g., larger font, converting text-to-speech, etc.) for a user of device component 202 that may be vision impaired.

The user-specific library interpretation component of the data distribution gateway 206 provides authorized data (i.e., authorized for view by the particular user) stored in the events log database to a user via a custom interface that is targeted toward that particular user. For example, if the user is a patient, a set of custom interfaces may be provided by the user-specific literacy interpretation component of the data distribution gateway 206, and if the user of device component 202 is a physician, another set of custom interfaces may be provided by the user-specific literacy interpretation component of the data distribution gateway 206. In the instance where the user is a layman user, the user-specific literacy interpretation component knows this is a patient (and they are not a healthcare professional), so they need layman's terminology. Accordingly, the user-specific literacy interpretation component may provide interfaces that provide a simple explanation of benefits to explain what a procedure was that the patient underwent, where such interfaces may even suggest any actions that they might want to take based on what they are viewing. For example, a user who wants to view their blood-pressure data may want to know if their value is too high or too low. The literacy interpretation component provides such additional context to the data being viewed depending on the type of user.

Additional interfaces that may be displayed via device component 202 may also be found in the concurrently filed non-provisional patent application titled “Graphical User Interfaces (GUIs) Associated with a Data Distribution Gateway of a Digital Healthcare Platform”, with Serial No. XX/XXX,XXX, which is fully incorporated by reference in its entirety.

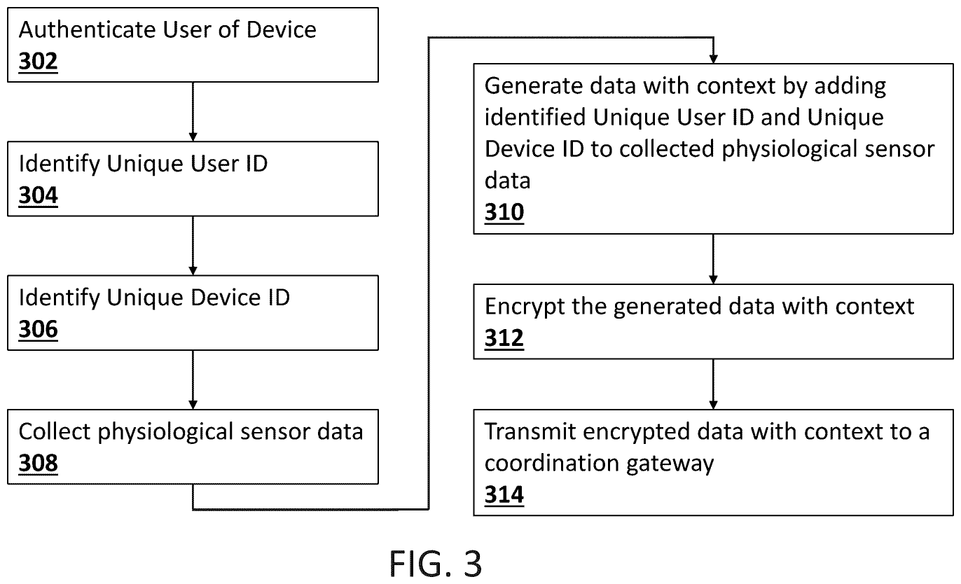

FIG. 3 depicts an example method as implemented in the device component of the digital healthcare framework. In step 302, the user of the device is authenticated. In step 304, the authenticated user's Unique User ID is identified and, in step 306, the device's Unique Device ID is identified. In step 308, physiological data is collected using one or more sensors in the device, and, in step 310, data with context is generated by adding the identified Unique User ID and the identified Unique Device ID to the collected physiological sensor data. In step 312, the generated data with context is encrypted by an encryption engine, and, in step 314, the encrypted data with context is transmitted to a coordination gateway.

In one embodiment, the present invention provides a medical device comprising: (a) a sensor configured to collect physiological data; (b) an authentication engine, the authentication engine configured to: (1) receive a unique device identifier (UDI), the UDI uniquely identifying the medical device; (2) receive a unique user identifier (UUI), the UUI uniquely identifying a user of the medical device; (3) authenticate the user as an authorized user of the medical device based on the UDI and the UUI; (4) upon successful authentication, formulate a data frame with: (i) collected physiological data, (ii) UUI, and (iii) UDI; (c) an encryption engine encrypting the data frame via an encryption algorithm; and (d) a transmission component transmitting the encrypted data frame.

In another embodiment, the present invention provides a method as implemented in a medical device, the method comprising: (a) collecting physiological data; (b) receiving a unique device identifier (UDI), the UDI uniquely identifying the medical device; (c) receiving a unique user identifier (UUI), the UUI uniquely identifying a user of the medical device; (d) authenticating the user as an authorized user of the medical device based on the UDI and the UUI; (e) upon successful authentication, formulating a data frame with: (1) collected physiological data, (2) UUI, and (3) UDI; (f) encrypting the data frame via an encryption algorithm; and (g) transmitting the encrypted data frame.

In yet another embodiment, the present invention provides an article of manufacture comprising non-transitory computer storage medium storing computer readable program code which, when executed by a computer, implements a method as implemented in a medical device, the medium comprising: (a) computer readable program code collecting physiological data; (b) computer readable program code receiving a unique device identifier (UDI), the UDI uniquely identifying the medical device; (c) computer readable program code receiving a unique user identifier (UUI), the UUI uniquely identifying a user of the medical device; (d) computer readable program code authenticating the user as an authorized user of the medical device based on the UDI and the UUI; (e) computer readable program code, upon successful authentication, formulating a data frame with: (1) collected physiological data, (2) UUI, and (3) UDI; (f) computer readable program code encrypting the data frame via an encryption algorithm; and (g) transmitting the encrypted data frame.

The above-described features and applications can be implemented as software processes that are specified as a set of instructions recorded on a computer readable storage medium (also referred to as computer readable medium). When these instructions are executed by one or more processing unit(s) (e.g., one or more processors, cores of processors, or other processing units), they cause the processing unit(s) to perform the actions indicated in the instructions. Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor. By way of example, and not limitation, such non-transitory computer-readable media can include flash memory, RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. The computer readable media does not include carrier waves and electronic signals passing wirelessly or over wired connections.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

In this specification, the term “software” is meant to include firmware residing in read-only memory or applications stored in magnetic storage or flash storage, for example, a solid-state drive, which can be read into memory for processing by a processor. Also, in some implementations, multiple software technologies can be implemented as sub-parts of a larger program while remaining distinct software technologies. In some implementations, multiple software technologies can also be implemented as separate programs. Finally, any combination of separate programs that together implement a software technology described here is within the scope of the subject technology. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

These functions described above can be implemented in digital electronic circuitry, in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, for example microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic or solid state hard drives, read-only and recordable BluRay® discs, ultra density optical discs, any other optical or magnetic media, and floppy disks. The computer-readable media can store a computer program that is executable by at least one processing unit and includes sets of instructions for performing various operations. Examples of computer programs or computer code include machine code, for example is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, for example application specific integrated circuits (ASICs) or field programmable gate arrays (FPGAs). In some implementations, such integrated circuits execute instructions that are stored on the circuit itself

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

It is understood that any specific order or hierarchy of steps in the processes disclosed is an illustration of example approaches. Based upon design preferences, it is understood that the specific order or hierarchy of steps in the processes may be rearranged, or that all illustrated steps be performed. Some of the steps may be performed simultaneously. For example, in certain circumstances, multitasking and parallel processing may be advantageous. Moreover, the separation of various system components illustrated above should not be understood as requiring such separation, and it should be understood that the described program components and systems can generally be integrated together in a single software product or packaged into multiple software products.

Various modifications to these aspects will be readily apparent, and the generic principles defined herein may be applied to other aspects. Thus, the claims are not intended to be limited to the aspects shown herein, but is to be accorded the full scope consistent with the language claims, where reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject technology.

A phrase, for example, an “aspect” does not imply that the aspect is essential to the subject technology or that the aspect applies to all configurations of the subject technology. A disclosure relating to an aspect may apply to all configurations, or one or more configurations. A phrase, for example, an aspect may refer to one or more aspects and vice versa. A phrase, for example, a “configuration” does not imply that such configuration is essential to the subject technology or that such configuration applies to all configurations of the subject technology. A disclosure relating to a configuration may apply to all configurations, or one or more configurations. A phrase, for example, a configuration may refer to one or more configurations and vice versa.

The various embodiments described above are provided by way of illustration only and should not be construed to limit the scope of the disclosure. Those skilled in the art will readily recognize various modifications and changes that may be made to the principles described herein without following the example embodiments and applications to illustrated and described herein, and without departing from the spirit and scope of the disclosure.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As noted above, particular embodiments of the subject matter have been described, but other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

### CONCLUSION

A system and method have been shown in the above embodiments for the effective implementation of a device component of a digital healthcare platform. While various preferred embodiments have been shown and described, it will be understood that there is no intent to limit the invention by such disclosure, but rather, it is intended to cover all modifications falling within the spirit and scope of the invention, as defined in the appended claims. For example, the present invention should not be limited by software/program, computing environment, or specific computing hardware.

1. A medical device comprising:
(a) a sensor configured to collect physiological data;
(b) an authentication engine, the authentication engine configured to:
(1) receive a unique device identifier (UDI), the UDI uniquely identifying the medical device;
(2) receive a unique user identifier (UUI), the UUI uniquely identifying a user of the medical device;
(3) authenticate the user as an authorized user of the medical device based on the UDI and the UUI;
(4) upon successful authentication, formulate a data frame with: (i) collected physiological data, (ii) UUI, and (iii) UDI;
(c) an encryption engine encrypting the data frame via an encryption algorithm; and
(d) a transmission component transmitting the encrypted data frame.

2. The medical device of claim 1, wherein the medical device is any of the following: a blood pressure monitor, glucometer, oximeter, spirometer, oxygen nebulizer, stethoscope, electrocardiogram (ECG) device, thermometer, activity tracker, or weighing scale.

3. The medical device of claim 1, wherein the UDI is hardcoded or encoded through SIM or eSIM type programmable technology.

4. The medical device of claim 1, wherein the UDI is information associated with hardware of the medical device.

5. The medical device of claim 4, wherein the information associated with hardware is any of the following: a model number of the medical device or a serial number of the medical device.

6. The medical device of claim 1, wherein the UDI is information associated with a software configuration of the medical device.

7. The medical device of claim 1, wherein the UDI is a host identifier assigned to the medical device by a registry function of a digital healthcare platform.

8. The medical device of claim 1, wherein the data frame further comprises any of, or a combination of, the following information: date, time, location information, and environmental information.

9. The medical device of claim 1, wherein the authorized user is authenticated via any of, or a combination of, the following: username and password combination, biometric authentication information, a keycard, a badge, two-factor authentication, a behavioral analysis of the user, or a virtual persona.

10. The medical device of claim 1, wherein the encryption algorithm is any of the following: Elliptic-curve Diffie-Hellman (ECDH), Edwards Elliptic Curve Digital Signature Algorithm (EdDSA) used in Host Identities (HI) and for Base Exchange (BEX) signatures, hashes used in Host Identity Tag (HIT) generation, keyed hashes used for KEYMAT (Keying Material) generation and packet MACing (Message Authentication Code) operations, and Authenticated Encryption with Associated Data (AEAD) and stream ciphers to use in Host Identify Protocol (HIP) and HIP enabled secure communication protocols.

11. A method as implemented in a medical device, the method comprising:
(a) collecting physiological data;
(b) receiving a unique device identifier (UDI), the UDI uniquely identifying the medical device;
(c) receiving a unique user identifier (UUI), the UUI uniquely identifying a user of the medical device;
(d) authenticating the user as an authorized user of the medical device based on the UDI and the UUI;
(e) upon successful authentication, formulating a data frame with: (1) collected physiological data, (2) UUI, and (3) UDI;
(f) encrypting the data frame via an encryption algorithm; and
(g) transmitting the encrypted data frame.

12. The method of claim 11, wherein the medical device is any of the following: a blood pressure monitor, glucometer, oximeter, spirometer, oxygen nebulizer, stethoscope, electrocardiogram (ECG) device, thermometer, activity tracker, or weighing scale.

13. The method of claim 11, wherein the UDI is hardcoded or encoded through SIM or eSIM type programmable technology.

14. The method of claim 11, wherein the UDI is information associated with hardware of the medical device, wherein the information associated with hardware is any of the following: a model number of the medical device or a serial number of the medical device.

15. The method of claim 11, wherein the UDI is information associated with a software configuration of the medical device.

16. The method of claim 11, wherein the UDI is a host identifier assigned to the medical device by a registry function of a digital healthcare platform.

17. The method of claim 11, wherein the data frame further comprises any of, or a combination of, the following information: date, time, location information, and environmental information.

18. The method of claim 11, wherein the authorized user is authenticated via any of, or a combination of, the following: username and password combination, biometric authentication information, a keycard, a badge, two-factor authentication, a behavioral analysis of the user, or a virtual persona.

19. The method of claim 11, wherein the encryption algorithm is any of the following: Elliptic-curve Diffie-Hellman (ECDH), Edwards Elliptic Curve Digital Signature Algorithm (EdDSA) used in Host Identities (HI) and for Base Exchange (BEX) signatures, hashes used in Host Identity Tag (HIT) generation, keyed hashes used for KEYMAT (Keying Material) generation and packet MACing (Message Authentication Code) operations, and Authenticated Encryption with Associated Data (AEAD) and stream ciphers to use in Host Identify Protocol (HIP) and HIP enabled secure communication protocols.

20. An article of manufacture comprising non-transitory computer storage medium storing computer readable program code which, when executed by a computer, implements a method as implemented in a medical device, the medium comprising:
(a) computer readable program code collecting physiological data;
(b) computer readable program code receiving a unique device identifier (UDI), the UDI uniquely identifying the medical device;
(c) computer readable program code receiving a unique user identifier (UUI), the UUI uniquely identifying a user of the medical device;
(d) computer readable program code authenticating the user as an authorized user of the medical device based on the UDI and the UUI;
(e) computer readable program code, upon successful authentication, formulating a data frame with: (1) collected physiological data, (2) UUI, and (3) UDI;
(f) computer readable program code encrypting the data frame via an encryption algorithm; and
(g) transmitting the encrypted data frame.
